(19) United States
(12) Patent Application Publication
(10) Pub. No.: US 2017/0196463 A1
(43) Pub. Date: Jul. 13, 2017

(54) PHYSIOLOGICAL PARAMETER PROCESSING APPARATUS, METHOD OF CONTROLLING OPERATION OF PHYSIOLOGICAL PARAMETER PROCESSING APPARATUS, AND PHYSIOLOGICAL PARAMETER PROCESSING SYSTEM

(71) Applicant: NIHON KOHDEN CORPORATION, Tokyo (JP)

(72) Inventors: Tetsuo SUZUKI, Tokyo (JP); Kazumasa ITO, Tokyo (JP)

(73) Assignee: NIHON KOHDEN CORPORATION, TOKYO (JP)

(21) Appl. No.: 15/389,702

(22) Filed: Dec. 23, 2016

(30) Foreign Application Priority Data
Jan. 8, 2016 (JP) 2016-002387

(51) Int. Cl. A61B 5/02

(52) U.S. Cl. CPC A61B 5/02

(57) ABSTRACT

A probe having a sensor configured to be attached to a living body to detect a physiological parameter and an information element configured to identify the sensor is connected to an interface of a physiological parameter processing apparatus. A detecting section of the physiological parameter processing apparatus detects, through the interface, whether the information element is present and whether the sensor is present. In response to the information element being detected but the sensor not being detected, a limiting processor of the physiological parameter processing apparatus limits at least a part of a function of the information element.

### CROSS-REFERENCE TO RELATED APPLICATION(S)

The present application claims priority from Japanese Patent Application No. 2016-002387 filed on Jan. 8, 2016, the entire content of which is incorporated herein by reference.

### BACKGROUND

The presently disclosed subject matter relates to a physiological parameter processing apparatus to which a probe is connectable, the probe including a sensor configured to be attached to a living body and an information element configured to identify the sensor. The presently disclosed subject matter also relates to a method of controlling an operation of the physiological parameter processing apparatus, and a physiological parameter processing system including the probe and the physiological parameter processing apparatus.

A pulse oximeter is an example of a physiological parameter processing apparatus to which a probe is connected. The probe to be connected to the pulse oximeter includes a light emitter and a light detector, an example of a sensor (see, e.g., JPH06-98881A).

Probes are disposable products, and counterfeit products are being distributed at lower prices. With a counterfeit probe, a physiological parameter may not be correctly acquired. Therefore, some genuine products includes an information element for authentication, so that when the probe is connected to a physiological parameter processing apparatus, the physiological parameter processing apparatus detects the information element to authenticate that the probe is a genuine product. Examples of such an information element include specific information stored in a memory and a specific circuit element.

However, there is a new way of counterfeiting. That is, a sensor is removed from a genuine probe such that a portion including an information element is left. One end (first end) of this portion is connected to a physiological parameter processing apparatus. The other end (second end) of this portion is connected to a connector. A disposable sensor is connected in a replaceable manner to the connector connected to the second end. Because the information element itself is genuine, it is possible to get through the authentication by the physiological parameter processing apparatus even with counterfeit sensors.

### SUMMARY

Illustrative aspects of the presently disclosed subject matter provide means that can prevent counterfeit sensors and probes from being used.

According to a first aspect of the presently disclosed subject matter, a physiological parameter processing apparatus to which a probe is connectable, the probe including a sensor configured to be attached to a living body to detect a physiological parameter and an information element configured to identify the sensor, is provided. The apparatus includes an interface to which the probe is connectable, a detecting section configured to detect, through the interface, whether the information element is present and whether the sensor is present, and a limiting processor configured to limit, in response to the information element being detected but the sensor not being detected, at least a part of a function of the information element.

According to a second aspect of the presently disclosed subject matter, a method of controlling an operation of a physiological parameter processing apparatus to which a probe is to be connected, the probe including a sensor configured to be attached to a living body to detect a physiological parameter and an information element configured to identify the sensor, is provided. The method includes causing the physiological parameter processing apparatus to detect through an interface to which the probe is connected, whether the information element is present and whether the sensor is present, and causing the physiological parameter processing apparatus to limit, in response to the information element being detected but the sensor not being detected, at least a part of a function of the information element.

According to a third aspect of the presently disclosed subject matter, a physiological parameter processing system includes a probe having a sensor configured to be attached to a living body to detect a physiological parameter and an information element configured to identify the sensor, an interface to which the probe is connectable, a detecting section configured to detect, through the interface, whether the information element is present and whether the sensor is present, and a limiting processor configured to limit, in response to the information element being detected but the sensor not being detected, at least a part of a function of the information element.

A genuine probe is configured such that a sensor and an information element are inseparable. Therefore, when only the information element is detected, there is a high likelihood that some kind of alteration is made on the genuine probe or a counterfeit probe is being used. In such a situation, by limiting at least the part of the function of the information element, the use of a counterfeit sensor or a counterfeit probe can be limited or prohibited. This also provides measures against the counterfeiting in which a sensor is removed from a genuine probe such that the information element is left and a counterfeit sensor is connected to the information element.

### BRIEF DESCRIPTION OF DRAWINGS

FIG. 1 illustrates a configuration of a physiological parameter processing system according to an exemplary embodiment of the presently disclosed subject matter;

FIGS. 2A to 2C illustrate an example of counterfeiting; and

FIGS. 3A to 3C illustrate examples of connection conditions between a probe and a physiological parameter processing apparatus of the physiological parameter processing system illustrated in FIG. 1.

### DETAILED DESCRIPTION

Hereinafter, exemplary embodiments of the presently disclosed subject matter will be described in detail with reference to the accompanying drawings. The drawings may not be to scale for the purpose of illustration.

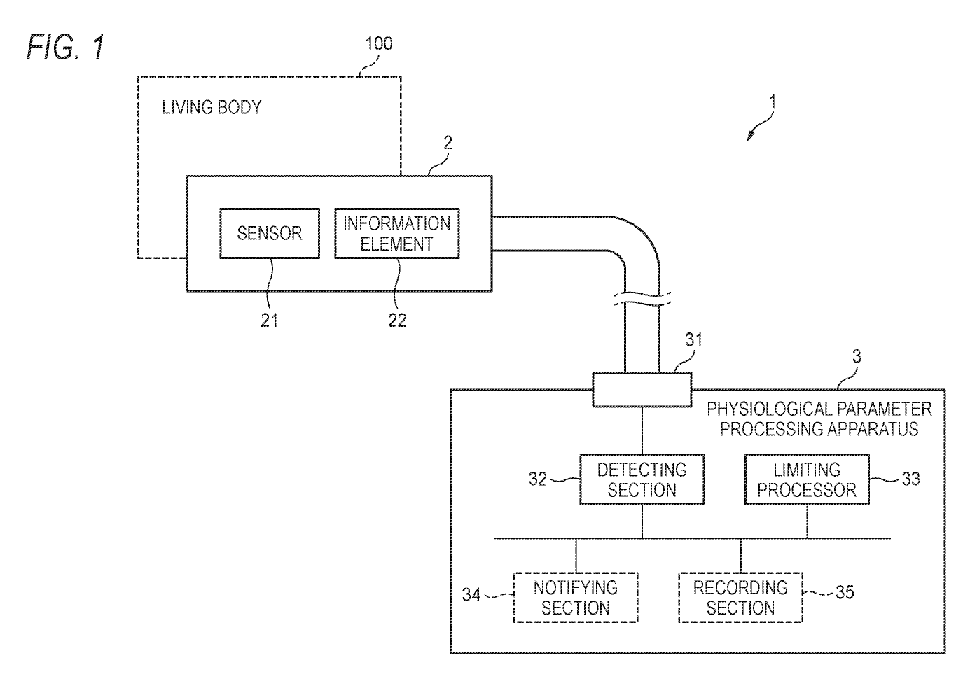

FIG. 1 illustrates a functional configuration of a physiological parameter processing system 1 according to an exemplary embodiment of the presently disclosed subject matter. The physiological parameter processing system 1 can include a probe 2 and a physiological parameter processing apparatus 3. The probe 2 can include a sensor 21 and an information element 22. The physiological parameter processing apparatus 3 can include an interface 31, a detecting section 32, and a limiting processor 33.

The sensor 21 is configured to be attached to a living body 100 to detect one or more physiological parameters. An example of the sensor 21 is an optical sensor having a light emitter and a light detector. With an optical sensor, blood light absorber concentration and pulse beats can be detected as physiological parameters. Another example of the sensor 21 is a pressure sensor such as a catheter configured to be inserted into a blood vessel and having a pressure transducer. With the catheter, blood pressure can be detected as a physiological parameter.

The information element 22 has information that identifies the sensor 21. Examples of the information element 22 can include a memory storing the information, a specific circuit element that shows a value corresponding to the information (e.g., the value of a resistance element, that of a capacitance element, that of an inductance element, or the forward drop voltage of a diode), and the like.

The interface 31 is configured such that the probe 2 is connectable to the interface 31. The detecting section 32 is configured to detect, through the interface 31, whether the sensor 21 is present and whether the information element 22 is present. With a memory being the information element 22, the detecting section 32 is configured to read information stored in the memory. With a circuit element being the information element 22, the detecting section 32 is configured to detect the presence of the information element 22 when a certain electrical response (a voltage value, a current value, or the like) is detected in response to an application of a certain voltage.

The physiological parameter processing apparatus 3 is configured such that, when the probe 2 is connected to the interface 31 and both the sensor 21 and the information element 22 are detected, the physiological parameter processing apparatus 3 authenticates that the connected probe 2 is a genuine product.

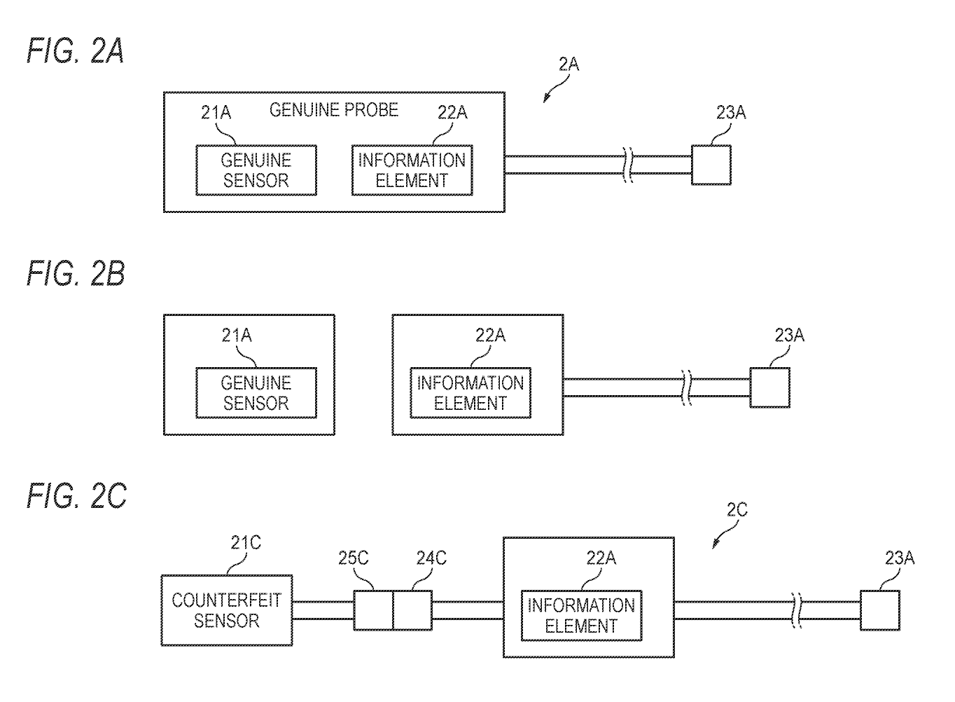

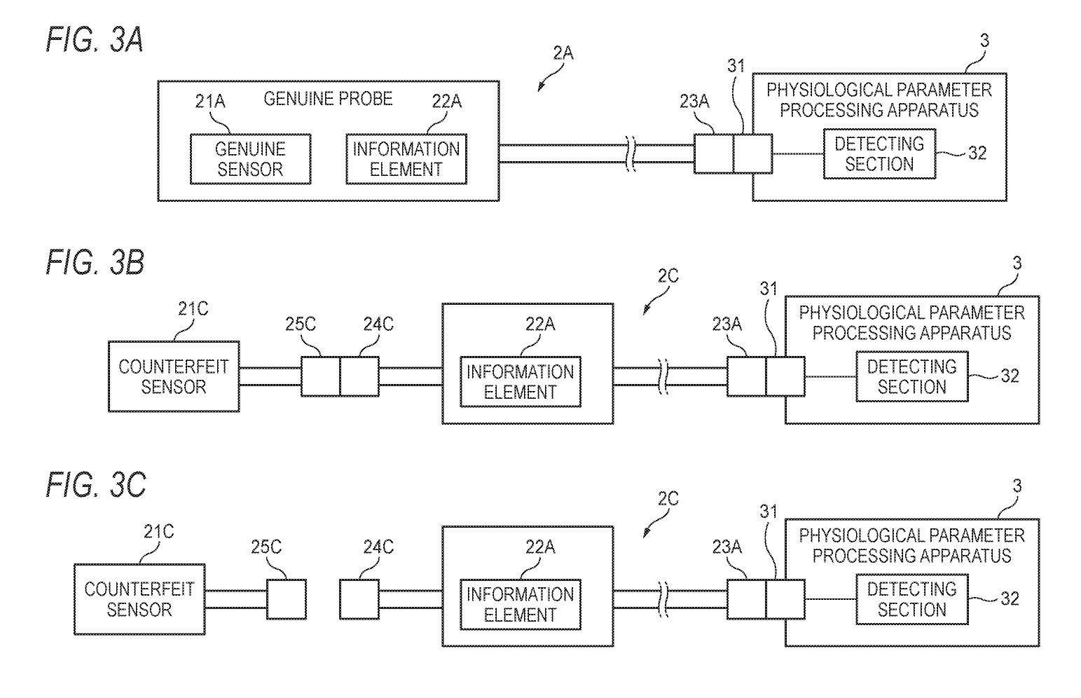

However, there is a way for counterfeiting using this authentication system. As illustrated in FIG. 2A, a genuine probe 2A always includes both a sensor 21A and an information element 22A. A connector 23A is configured so as to be connected to the interface 31 of the physiological parameter processing apparatus 3. As illustrated in FIG. 3A, when both the sensor 21A and the information element 22A are detected, the physiological parameter processing apparatus 3 authenticates that the connected probe 24 is a genuine product.

As illustrated in FIG. 2B, a counterfeit provider removes the sensor 21A from the probe 2A such that a portion including the information element 22A and the connector 23A is left. Then, as illustrated in FIG. 2C, a separately prepared connector 24C is connected to the portion including the information element 22A. The information element 22A and the connector 24C are electrically connected to each other. In this way, the counterfeit provider provides the altered part and the counterfeit sensor 21C with a connector 25C.

The connector 24C and the connector 25C are connected to each other, and a counterfeit probe 2C is completed. When the connector 23A (genuine portion) of the counterfeit probe 2C is connected to the interface 31 of the physiological parameter processing apparatus 3, as illustrated in FIG. 3B, the detecting section 32 detects both the counterfeit sensor 21C and the information element 22A (genuine portion). Therefore, the counterfeit probe 2C is authenticated as a genuine probe. The user continuously obtains only counterfeit sensors 21C, and repeats the connection and disconnection of the connector 24C and the connector 25C.

The limiting processor 33 of the physiological parameter processing apparatus 3 illustrated in FIG. 1 is configured to limit, in response to the information element 22 being detected but the sensor 21 not being detected, limit at least a part of the function of the information element 22.

As illustrated in FIG. 3C, the user of the counterfeit probe 2C disconnects the connector 24C and the connector 25C to replace the counterfeit sensor 21C which is a disposable product. In this state, there is temporarily a condition where the information element 22A is detected but the counterfeit sensor 21C is not detected. When this condition is detected, the physiological parameter processing apparatus 3 detects that there is some kind of alteration made on the probe 2 or the counterfeit probe 2C is being used. The genuine probe 2A illustrated in FIG. 2A is configured such that the sensor 21A and the information element 22A are inseparable from each other. In the genuine probe, therefore, such a condition does not occur.

When the information element 22A is a memory, the limiting processor 33 writes data for limiting the use of the counterfeit sensor 21C, in the memory. When, as a result of replacement, the counterfeit sensor 21C is connected to the portion including the information element 22A, the detecting section 32 of the physiological parameter processing apparatus 3 reads the data which are written in the memory, and which relate to the use of a counterfeit product. Therefore, the authentication which should be performed in the case of the connection of the genuine probe 2 is not conducted, and the use of the counterfeit sensor 21C and the counterfeit probe 2C can be limited or prohibited.

When the information element 22A is a circuit element such as a resistor, a capacitor, an inductor, a diode, or a transistor, the limiting processor 33 applies an overvoltage or overcurrent to the circuit element to electrically break the circuit element. Even when, as a result of replacement, a new counterfeit sensor 21C is connected to the portion including the information element 22A, the detecting section 32 of the physiological parameter processing apparatus 3 does not detect the information element 22A. Therefore, the authentication which should be performed in the case of the connection of the genuine probe 2 is not conducted, and the use of the counterfeit sensor 21C and the counterfeit probe 2C can be prohibited.

When the information element 22A is a memory, the limiting processor 33 may apply an overvoltage or overcurrent to the memory to electrically break the memory.

As illustrated by the broken line in FIG. 1, the physiological parameter processing apparatus 3 may include a notifying section 34. The notifying section 34 is configured to notify that at least a part of the function of the information element 22 is limited by the limiting processor 33. The notification may be visually performed through a displaying section or the like, or may be audibly performed by a spoken message, an alarm sound, or the like.

According to this configuration, in a situation where the counterfeit probe 2C is inoperable as a result of the authentication, the user can recognize that the current situation is caused by the use of the counterfeit probe 2C, without suspecting a failure of the physiological parameter processing apparatus 3.

As illustrated by the broken line in FIG. 1, the physiological parameter processing apparatus 3 may include a recording section 35. The recording section 35 records, as history information, a fact that at least a part of the function of the information element 22 is limited by the limiting processor 33.

According to the configuration, when the history information recorded in the recording section 35 is checked during maintenance or the like of the physiological parameter processing apparatus 3, it is possible to confirm the fact that the user has used the counterfeit probe 2C. By informing every user that history information is recorded as described above, it is also possible to prevent a use of the counterfeit probe 2C in advance.

When the use of the counterfeit probe 2C is detected by the detecting section 32, the physiological parameter processing apparatus 3 may record the result of the detection by the detecting section 32 as history information in the recording section 35, instead of limiting at least a part of the function of the information element 22 by the limiting processor 33. This also makes it possible to later confirm the fact that the user has used the counterfeit probe 2C.

The functions of the detecting section 32, the limiting processor 33, the notifying section 34, and the recording section 35 may be implemented by a software executed by a cooperation of a processor and a memory that are connected to each other to allow communication. Examples of the processor include a CPU and an MPU. Examples of the memory include a RAM and a ROM. At least one of the functions of the detecting section 32, the limiting processor 33, the notifying section 34, and the recording section 35 may be implemented by hardware such as circuit devices, or a combination of hardware and software.

While the presently disclosed subject matter has been described with reference to certain exemplary embodiments thereof, the scope of the presently disclosed subject matter is not limited to the exemplary embodiments described above, and it will be understood by those skilled in the art that various changes and modifications may be made therein without departing from the scope of the presently disclosed subject matter as defined by the appended claims.

What is claimed is:

1. A physiological parameter processing apparatus to which a probe is connectable, the probe including a sensor configured to be attached to a living body to detect a physiological parameter and an information element configured to identify the sensor, the apparatus comprising:
an interface to which the probe is connectable;
a detecting section configured to detect, through the interface, whether the information element is present and whether the sensor is present; and
a limiting processor configured to limit, in response to the information element being detected but the sensor not being detected, at least a part of a function of the information element.

2. The apparatus according to claim 1, wherein the information element is a memory, and wherein the limiting processor is configured to write, in the memory, data for limiting a use of the sensor.

3. The apparatus according to claim 1, wherein the information element is a circuit element, and wherein the limiting processor is configured to electrically break the circuit element.

4. The apparatus according to claim 1, further comprising a notifying section configured to notify that at least the part of the function of the information element is limited by the limiting processor.

5. The apparatus according to claim 1, further comprising a recording section configured to record, as history information, a fact that at least the part of the function of the information element has been limited by the limiting processor.

6. The apparatus according to claim 1, further comprising a recording section configured to record, as history information, a result of a detection by the detecting section, the result including the information element being detected but the sensor not being detected

7. The apparatus according to claim 1, wherein the interface is configured such that the probe including the sensor, the sensor having a light emitter and a light detector or the sensor being a pressure sensor, is connectable to the interface.

8. A method of controlling an operation of a physiological parameter processing apparatus to which a probe is to be connected, the probe including a sensor configured to be attached to a living body to detect a physiological parameter and an information element configured to identify the sensor, the method comprising:
causing the physiological parameter processing apparatus to detect through an interface to which the probe is connected, whether the information element is present and whether the sensor is present; and
causing the physiological parameter processing apparatus to limit, in response to the information element being detected but the sensor not being detected, at least a part of a function of the information element.

9. The method according to claim 8, wherein the information element is a memory, and wherein the causing the physiological parameter processing apparatus to limit at least the part of the function of the information element comprises writing, in the memory, data for limiting a use of the sensor.

10. The method according to claim 8, wherein the information element is a circuit element, and wherein the causing the physiological parameter processing apparatus to limit at least the part of the function of the information element comprises electrically breaking the circuit element.

11. The method according to claim 8, further comprising causing the physiological parameter processing apparatus to notify that at least the part of the function of the information element is limited.

12. The method according to claim 8, further comprising causing the physiological parameter processing apparatus to record, as history information, a fact that at least the part of the function of the information element has been limited.

13. A physiological parameter processing system comprising:
a probe comprising a sensor configured to be attached to a living body to detect a physiological parameter and an information element configured to identify the sensor;
an interface to which the probe is connectable;
a detecting section configured to detect, through the interface, whether the information element is present and whether the sensor is present; and
a limiting processor configured to limit, in response to the information element being detected but the sensor not being detected, at least a part of a function of the information element.

14. The system according to claim 13, wherein the information element is a memory, and wherein the limiting processor is configured to write, in the memory, data for limiting a use of the sensor.

15. The system according to claim 13, wherein the information element is a circuit element, and wherein the limiting processor is configured to electrically break the circuit element.

16. The system according to claim 13, further comprising a notifying section configured to notify that at least the part of the function of the information element is limited by the limiting processor.

17. The system according to claim 13, further comprising a recording section configured to record, as history information, a fact that at least the part of the function of the information element has been limited by the limiting processor.
